(19) United States
(12) Patent Application Publication
(10) Pub. No.: US 2005/0154343 A1
(43) Pub. Date: Jul. 14, 2005

(54) Soft tip applicator for relieving mouth pain

(76) Inventor: Mohinder Singh, Naperville, IL (US)

Correspondence Address: LADAS & PARRY LLP, 224 SOUTH MICHIGAN AVENUE, SUITE 1200, CHICAGO, IL, 60604, US

(21) Appl. No.: 10/757,196

(22) Filed: Jan. 14, 2004

(51) Int. Cl. A61M 35/00; A61K 9/00

(52) U.S. Cl. 604/2

(57) ABSTRACT

An applicator for treating mouth pain includes a generally cylindrical container having a fluid reservoir therein, a dispensing orifice at one end of the container in fluid communication with the reservoir, a plurality of soft bristles protruding outwardly from the orifice acting as a partial closure for the orifice and which act as an applicator for fluid in the reservoir, means for forcing fluid from the reservoir to the bristles, and a composition contained in the reservoir for treating mouth pain. A method of applying a fluid composition for treating mouth pain includes the use of the applicator with the fluid composition for direct application to all areas of the mouth.

### BACKGROUND OF THE INVENTION

The invention relates to a method and apparatus for dispensing a topical anesthetic to areas of the human mouth to relieve pain.

Mouth pain can originate from a variety of sources, including canker sores, toothache, irritation from braces or dentures, hot food burns, cuts inside the mouth and irritation or infection of the gums. Topical anesthetics have long been used to relieve mouth pain, typically in the form of a liquid or gel which is applied in the mouth by dispensing from a tube onto a finger. A liquid composition may also be used which is dispensed from a container used a plastic rod attached to the container cap and which is applied directly onto the sore area of the mouth.

Dentists use topical anesthetics to numb the mouth, often before a needle injection. In this case, the anesthetic is applied from a cotton or foam swab, dipped into a container of the anesthetic composition. Variations of this type of application which are suitable for dentists' use can be found in U.S. Pat. Nos. 5,016,651 and 5,762,494.

A more general type of anesthetic swab can be found in U.S. Pat. No. 5,704,906, in which the swab and an anesthetic-cleaning solution are contained in a flexible package which is torn open for a single use.

U.S. Pat. No. 6,358,231 discloses a dispenser for anesthetizing the ear canal including a long neck container with a fluid reservoir at one end of the neck and a sponge applicator releasably secured to the other end of the neck. The sponge applicator may be wetted by squeezing the reservoir, and then inserted into the ear and further wetted. The container is finally detached from the applicator, which remains in the ear canal. Each sponge applicator is thus used for a single application only, although another applicator may be attached to the container if enough fluid remains for further application therefrom. While the structure shown in this patent is useful for treating the ear canal, it is not useful for application to other areas of the body, particularly the mouth, where it is not desired that the applicator be detached, and where a number of applications of anesthetic composition would be desired.

### SUMMARY OF THE INVENTION

It is therefore an object of the invention to provide an applicator suitable for multiple applications of an anesthetic composition to the mouth to treat sore areas therein.

It is a further object of the invention to provide an applicator with a soft tip such that sore areas are not further irritated by application of the anesthetic composition.

It is another object of the present invention to provide an application of sufficient size to reach all areas within the mouth.

It is another object of the present invention to provide a method of applying a fluid composition to the mouth to relieve pain.

To achieve these and other objects, the invention is directed to the combination of an anesthetic composition for application to the mouth and an apparatus for dispensing the composition, comprising a generally cylindrical container having a fluid reservoir therein, a dispensing orifice at one end of the container in fluid communication with the reservoir, a plurality of soft bristles protruding outwardly from the orifice acting as a partial closure for the orifice and which act as an applicator for fluid in the reservoir, and means for forcing fluid from the reservoir to the bristles.

The dispenser is preferably a dispenser as disclosed in U.S. Pat. No. 6,227,739 which is incorporated herein by reference, although other similar dispensers may also be used.

The dispenser shown in U.S. Pat. No. 6,227,739 includes a body having a tank portion for housing a liquid, and a liquid supply port at a front side thereof, a piston movable forwardly inside the tank portion, an operation cylinder being attached to a rear part of the body in a relatively rotatable fashion, a piston rod being disposed within an assembly comprising the body and the operation cylinder, the piston rod integrally connected to the piston and extending rearwardly, the piston rod being disposed within the assembly and adapted to be rotated integrally with the operation cylinder. The piston rod guide has an internal thread hole which is engaged with the external thread of the piston rod, and a ratchet cylinder is fixed in the rear inside of the body and has a bore through which the piston rod is pierced in a relatively unrotatable fashion. Serrated gear teeth are formed at a front end of the operation cylinder, and the ratchet gear tooth, which is brought into engagement with the serrated gear teeth and can selectively protrude or retract in the axial direction. The ratchet gear tooth is adopted to be selectively protruded or retracted in an axial direction upon the rotation of the operation cylinder relative to the body. When the operation cylinder rotates relative to the body, the piston rod guide rotates integrally with the operation cylinder, while the piston rod does not rotate. A relative rotation takes place between the piston rod guide and the piston rod so that thread engagement between the internal thread hole of the piston rod guide and the external thread of the piston rod causes the piston rod to move forwardly in the tank portion, and the liquid inside the tank portion is pushed into the liquid supply port provided at the tip side of the body.

According to the invention, the anesthetic composition is preferably a clear gel containing no sugar, so it can be used by diabetics. The preferred composition also contains no saccharine or cyclamates. A preferred composition contains, by weight:

| | |
|---|---|
| benzocaine | 5-20% |
| polyethylene glycol | 5-60% |
| glycerin | 0.5-10% |
| flavoring | 0.1-5% |
| silica | 0.001-3% |
| sucralose sweetener | 0.1-3% |
| preservatives | 0.1-3% |

The polyethylene glycol can have a chain length in the range of 4 to 800, depending on the desired consistency of the composition, and the silica can be in the form of amorphous silica, fumed silica or spherons. The product is formulated with a viscosity sufficient that it will not leak from the bristles in the warmest weather expected to be encountered.

The container has dimensions selected so that it can fit easily into the mouth and can easily be carried in a pocket or purse. A preferred size is a length of 4.5 inches and a diameter of 0.5 inches, although other convenient sizes may also be used, for example a length of 1.5 to 6 inches, and a diameter of 0.25 to 0.75 inches.

### BRIEF DESCRIPTION OF THE DRAWINGS

FIG. 1 is a lateral view of a dispenser according to the invention; and

FIG. 2 is a lateral cross-sectional view of a dispenser according to the invention.

### DESCRIPTION OF THE PREFERRED EMBODIMENT

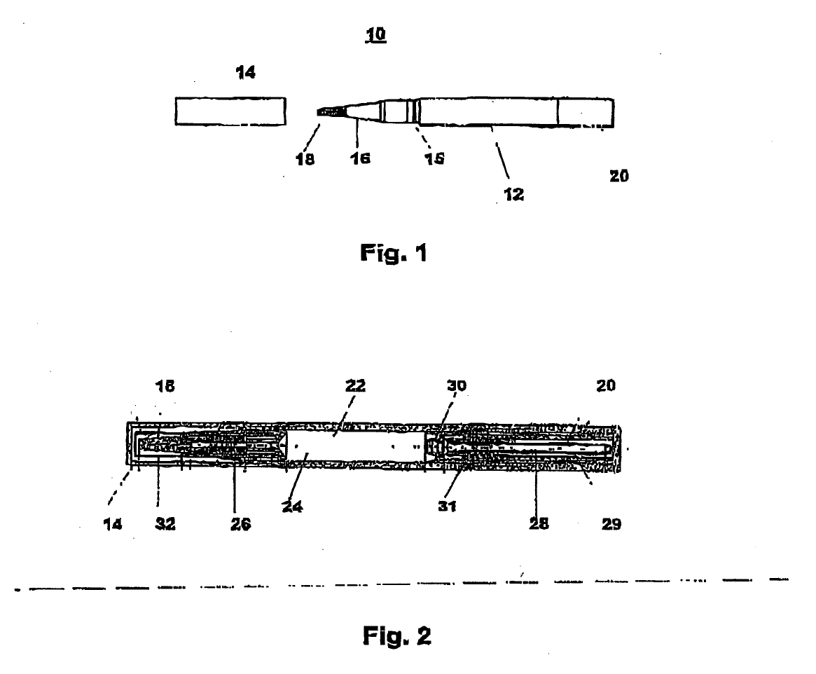

A preferred dispenser 10 according to the invention is shown in FIG. 1. The dispenser includes a main body portion 12 and a cap 14 which fits onto shoulder 15 of a tapered neck 16 at a front portion of the dispenser. Soft bristles 18 extend from neck 16 for application of the fluid contained in the dispenser by twisting cylinder 20 disposed at the rear portion of the dispenser.

The mechanism of the dispenser is shown in FIG. 2. A centrally located reservoir 22 contains the composition of the invention 24 which is in the form of a gel or fluid composition. The gel 24 is forced through an orifice 26 by rotation of cylinder 20, which forces a piston rod 28 of piston 30 towards the front portion of the dispenser, putting pressure on the reservoir 24. The piston rod 28 typically carries an external screw thread 29 which interacts with an internal screw thread in piston rod guide 31, to cause the forward movement of the piston upon rotation of the cylinder 20.

The viscosity of the fluid or gel in the reservoir prevents leakage of the fluid without the application of pressure to the reservoir. Further, the viscosity of the gel allows it to be sufficiently soft to prevent caking between soft bristles 18. In addition, an inner cap 32 protects the brush bristles 18.

### EXAMPLE

A composition was formulated containing, in % by weight:

| | |
|---|---|
| Benzocaine | 20.000 |
| polyethylene glycol-8 | 49.699 |
| polyethylene glycol-75 | 25.300 |
| Glycerin | 1.900 |
| sucralose (1,6-dichloro-1,6- | 0.900 |
| deoxy-β-D-fructofuranosyl-4- | |
| chloro-4-deoxy-α-D- | |
| galactopyranoside) | |
| propylparaben | 0.300 |
| methylparaben | 0.300 |
| ethylparaben | 0.200 |
| butylparaben | 0.200 |
| cherry flavor | 1.000 |
| mint flavor | 0.200 |
| silica | 0.001 |

The method of applying a sugar free fluid composition to a human mouth includes the use of the applicator and fluid composition described herein. The method involves using a generally cylindrical container having a fluid composition reservoir therein a dispensing orifice at one end of the container in fluid communication with the reservoir, a plurality of soft bristles protruding outwardly from the orifice acting as a partial closure for the orifice and which acts as an application for fluid composition in the reservoir, with means for forcing the fluid composition from the reservoir to the bristles, comprising the steps of rotating a cylinder at an end of the container opposite the bristles to activate the means for forcing the fluid composition from the reservoir through the bristles into the mouth.

The method further involves the use of means for forcing fluid from the reservoir comprising a piston disposed at an end of the container opposite to the orifice, a piston rod connected to the piston and directed away from the reservoir, and a rotatable cylinder at the end of the container opposite to the bristles wherein rotating the cylinder causes movement of the piston rod and piston towards the reservoir to force the fluid composition from the reservoir through the bristles into the mouth.

The foregoing description and examples have been set forth merely to illustrate the invention and are not intended to be limiting. Since modification of the described embodiments incorporating the spirit and substance of the invention may occur to persons skilled in the art, all such modifications are intended to be included with the scope of the claims.

1. An applicator for treating mouth pain, comprising:
a generally cylindrical container having a fluid reservoir therein,
a dispensing orifice at one end of the container in fluid communication with the reservoir,
a plurality of soft bristles protruding outwardly from the orifice acting as a partial closure for the orifice and which act as an applicator for fluid in the reservoir,
means for forcing fluid from the reservoir to the bristles, and
a composition contained in the reservoir for treating mouth pain.

2. The applicator of claim 1, wherein the composition comprises, by weight:

| | |
|---|---|
| benzocaine | 5-20% |
| polyethylene glycol | 5-60% |
| glycerin | 0.5-10%. |

3. The applicator of claim 2, wherein the composition further comprises, by weight:

| | |
|---|---|
| flavoring | 0.1-5% |
| silica | 0.001-3% |
| sucralose sweetener | 0.1-3% |
| preservative | 0.1-3%. |

4. The applicator of claim 1, wherein the composition comprises, in % by weight:

| | |
|---|---|
| benzocaine | 20.000 |
| polyethylene glycol-8 | 49.699 |
| polyethylene glycol-75 | 25.300 |
| glycerin | 1.900 |
| sucralose (1,6- | 0.900 |
| dichloro-1,6-deoxy-β- | |
| D-fructofuranosyl-4- | |
| chloro-4-deoxy-α-D- | |
| galactopyranoside) | |
| propylparaben | 0.300 |
| methylparaben | 0.300 |
| ethylparaben | 0.200 |
| butylparaben | 0.200 |
| cherry flavor | 1.000 |
| mint flavor | 0.200 |
| silica | 0.001 |

5. The applicator of claim 1, wherein the means for forcing fluid from the reservoir comprises a piston disposed at an end of the container opposite to the orifice, a piston rod connected to the piston and directed away from the reservoir, and a cylinder at an end of the container opposite to the bristles, which is rotatable to cause movement of the piston rod and piston towards the reservoir to force fluid therefrom.

6. The application of claim 1, wherein the viscosity of the composition is sufficient to prevent dripping of the composition from the dispensing orifice after use and to prevent caking of the composition between the bristles.

7. A method of applying to a human mouth a sugar free fluid composition for treating pain in the mouth
using a generally cylindrical container having a fluid composition reservoir therein;
a dispensing orifice at one end of the container in fluid communication with the reservoir
a plurality of soft bristles protruding outwardly from the orifice acting as a partial closure for the orifice and which acts as an application for fluid composition in the reservoir, with means for forcing the fluid composition from the reservoir to the bristles comprising the steps of
rotating a cylinder at an end of the container opposite the bristles to activate the means for forcing the fluid composition from the reservoir through the bristles into the mouth.

8. The method of claim 1 wherein the means for forcing fluid from the reservoir comprises a piston disposed at an end of the container opposite to the orifice, a piston rod connected to the piston and directed away from the reservoir, and a rotatable cylinder at the end of the container opposite to the bristles wherein rotating the cylinder causes movement of the piston rod and piston towards the reservoir to force the fluid composition from the reservoir through the bristles into the mouth.

9. The method of claim 7 wherein the fluid composition comprises by weight:

| | |
|---|---|
| benzocaine | 5-20% |
| polyethylene glycol | 5-60% |
| glycerin | 0.5-10%. |
